(19) United States
(12) Patent Application Publication
(10) Pub. No.: US 2022/0091100 A1
(43) Pub. Date: Mar. 24, 2022

(54) METHOD FOR MEASUREMENT OF LIVE-CELL PARAMETERS FOLLOWED BY MEASUREMENT OF GENE AND PROTEIN EXPRESSION

(71) Applicant: Arizona Board of Regents on behalf of Arizona State University, Scottsdale, AZ (US)

(72) Inventors: Clifford Anderson, Tempe, AZ (US); Dmitry Derkach, Gilbert, AZ (US); Deirdre Meldrum, Phoenix, AZ (US); Laimonas Kelbauskas, Gilbert, AZ (US)

(21) Appl. No.: 17/538,353

(22) Filed: Nov. 30, 2021

Related U.S. Application Data
(63) Continuation of application No. 15/774,563, filed on May 8, 2018, now abandoned.
(60) Provisional application No. 62/255,883, filed on Nov. 16, 2015.

(51) Int. Cl. G01N 33/50; C12Q 1/686; G01N 21/64

(52) U.S. Cl. CPC G01N 33/5005; G01N 21/6486; G01N 21/6452; C12Q 1/686

(57) ABSTRACT

A method for analyzing cells through measurement of live-cell parameters followed by measurement of gene and protein expression is disclosed herein. The method comprises measuring one or more live-cell parameters for a plurality of cells contained in at least one liquid in a plurality of isolated microchambers of a microarray device. The method further comprises removing a lid bounding the plurality of isolated microchambers. The method further comprises microdispensing a quantity of lysate into each microchamber of the plurality of isolated microchambers. The method further comprises microdispensing a quantity of reverse transcription polymerase chain reaction mix into each microchamber of the plurality of isolated microchambers. The method further comprises microdispensing a quantity of oil into each microchamber of the plurality of isolated microchambers. The method further comprises incorporating the microarray device into a thermal cycling apparatus with a window permitting epifluorescence imaging of the plurality of isolated microchambers.

### STATEMENT OF RELATED APPLICATION(S)

This application is a continuation of U.S. patent application Ser. No. 15/774,563 filed on May 8, 2018, which is the U.S. national phase under 35 U.S.C. § 371 of International Application No. PCT/US2016/062208 filed on Nov. 16, 2016, which claims the benefit of U.S. Provisional Patent Application No. 62/255,883 filed Nov. 16, 2015, wherein the entire disclosures of the foregoing applications are hereby incorporated by reference herein.

### GOVERNMENT RIGHTS IN INVENTION

This invention was made with government support under U01 CA164250 and P50 HG002360 awarded by the National Institutes of Health. The government has certain rights in the invention.

### TECHNICAL FIELD

The disclosure relates to a method for high-throughput, sequential measurement of live-cell parameters in isolated microchambers (e.g., “microwells”) followed by quantitative Reverse Transcription PCR, which maintains independence between results for different microchambers.

### BACKGROUND

Measurement of live-cell parameters in isolated microchambers is well-established. See, e.g., Kelbauskas, et al., “Method for physiologic phenotype characterization at the single-cell level in non-interacting and interacting cells,” J. Biomed. Opt., 17, 037008 (2012). qRT-PCR [real-time RT (reverse transcription)-PCR] has become a standard for the detection and quantification of RNA targets. qRT-PCR assays utilize fluorescent reporter molecules to monitor production of amplification products during each polymerase chain reaction cycle, and combine amplification and detection steps. Fluorescence-based qRT-PCR realizes the inherent quantitative capacity of PCR-based assays. Single-cell qRT-PCR has already been described. See, e.g., International Patent Application Publication No. WO/2015/048009; Beer, et al., “On-Chip Single-Copy Real-Time Reverse-Transcription PCR in Isolated Picoliter Droplets,” Anal. Chem., 80, 1854-1858 (2008). High-throughput non-contact dispensing of small volume droplets is well established, such as shown in the Rainmaker MicroDispensing Pattern Generator by Engineering Arts. The use of one-step qRT-PCR, the use of mineral oil for minimizing evaporation in biosciences applications, and thermal cycling apparatus design for glass substrates for qRT-PCR are also well established. Harvesting of single cells from microchambers after metabolic measurement for the purpose of downstream qRT-PCR has been demonstrated. See, e.g., Zeng, et al., “Quantitative single-cell gene expression measurements of multiple genes in response to hypoxia treatment,” Anal. Bioanal. Chem., 401, 3-13 (2011). Other methods have been demonstrated for nanoliter volume, high-throughput qRT-PCR with bulk cell samples, but not for single cell samples. See, e.g., Morrison, et al., “Nanoliter high throughput quantitative PCR,” Nucleic Acids Research, Vol. 34, No. 18 (2006); Zhang, et al., “Miniaturized PCR chips for nucleic acid amplification and analysis: latest advances and future trends,” Nucleic Acids Research, Vol. 35, No. 13, 4223-4237 (2007); Dittrich, et al., “Micro Total Analysis Systems. Latest Advancements and Trends,” Anal. Chem. 78, 3887 (2006); and Lee, et al., “A Disposable Plastic-Silicon Micro PCR Chip Using Flexible Printed Circuit Board Protocols and Its Application to Genomic DNA Amplification.” IEEE Sensors J., Vol. 8, No. 5 (2008).

The art continues to seek a method for harvesting of single cells for gene expression analysis that is high-throughput, is not operator dependent, does not pose a risk of degradation of RNA during transport of the cell, and preferably uses equipment that is commercially available. Also, the speed of conventional cell harvesting processes is slow, which can lead to long dwell times that can bias qRT-PCR results. In the harvesting context, it is also difficult to verify dispensation of a single cell into lysis buffer, and performance of verification steps reduces throughput. Such matter disclosed herein addresses (e.g., eliminates or substantially resolves) some or all of the foregoing concerns.

### SUMMARY

This invention disclosure describes a method for high-throughput, sequential measurement of live-cell parameters in isolated microchambers (e.g., “microwells”, or simply “wells”) followed by one-step, quantitative Reverse Transcription PCR (qRT-PCR) which maintains independence between results for different microchambers. The method consists of live-cell microchamber measurements; removal of the microchamber lid; optional removal of a portion of microchamber fluid using evaporation, blotting, or gas flow; optional dispensation of control RNA into a subset of microchambers; microdispensation of a droplet of lysate into each microchamber; microdispensation of a droplet of RT-PCR mix into each microchamber whereby primers may be different between microchambers; microdispensation of a droplet of oil to cap each microchamber and prevent evaporation; optional application of a lid to further prevent evaporation; and incorporation of the microchamber substrate into a thermal cycling apparatus with a window, thereby enabling epifluorescence imaging. A gene or protein expression assay can be run at the single-cell level using cells that were already monitored for metabolic parameters. Correlation is possible between metabolic and gene or protein expression parameters at the single cell level and at high-throughput and relatively lower cost.

In certain aspects, the present disclosure relates to a method for analyzing cells, the method comprising: measuring one or more live-cell parameters (e.g., oxygen concentration, oxygen consumption rate, pH, glucose concentration, glucose consumption rate, adenosine triphosphate (ATP) concentration, and/or mitochondrial membrane potential (MMP)) for a plurality of cells contained in at least one liquid in a plurality of isolated microchambers of a microarray device; removing a lid bounding the plurality of isolated microchambers; microdispensing a quantity of lysate into each microchamber of the plurality of isolated microchambers; microdispensing a quantity of reverse transcription polymerase chain reaction mix into each microchamber of the plurality of isolated microchambers; microdispensing a quantity of oil into each microchamber of the plurality of isolated microchambers; and incorporating the microarray device into a thermal cycling apparatus with a window permitting epifluorescence imaging of the plurality of isolated microchambers. In certain embodiments, each microchamber of the plurality of isolated microchambers comprises a volume in a range of from about 100 picoliters (pL) to about 500 picoliters (pL). In certain embodiments, said removing of the lid bounding the plurality of isolated microchambers causes removal of a portion of the at least one liquid from the plurality of isolated microchambers. In certain embodiments, the method further comprises removing a portion of the at least one liquid from the plurality of isolated microchambers by at least one of evaporation, blotting, or application of a gas flow. In certain embodiments, the method further comprises microdispensing a quantity of control RNA into a subset of microchambers of the plurality of isolated microchambers. In certain embodiments, at least one of said microdispensing of a quantity of lysate, microdispensing of a quantity of reverse transcription polymerase chain reaction mix, or microdispensing of a quantity of oil comprises piezoelectric microdispensing. In certain embodiments, at least one of said quantity of lysate, said quantity of reverse transcription polymerase chain reaction mix, or said quantity of oil comprises a volume in a range of from about 25 pL to about 200 pL, or in a range of from about 50 pL to about 150 pL. In certain embodiments, each microchamber of the plurality of isolated microchambers contains a single cell of the plurality of cells. In certain embodiments, the measuring of one or more live-cell parameters is performed at a single-cell level. In certain embodiments, the method further comprises performing a reverse transcription polymerase chain reaction in each microchamber of the plurality of isolated microchambers. In certain embodiments, the method further comprises performing protein expression measurement at a single-cell level in each microchamber of the plurality of isolated microchambers. In certain embodiments, the method utilizes a fixture incorporating at least one piezoelectric dispensing head and incorporating an apparatus for measuring fluorescence response at a single-cell level for each microchamber of the plurality of isolated microchambers. In certain embodiments, the method utilizes a fixture incorporating the thermal cycling apparatus and an apparatus for measuring fluorescence response for each microchamber of the plurality of isolated microchambers. In certain embodiments, said one or more live-cell parameters comprises at least one of oxygen concentration, oxygen consumption rate, pH, glucose concentration, glucose consumption rate, adenosine triphosphate concentration, or mitochondrial membrane potential. In certain embodiments, said quantity of lysate and said quantity of reverse transcription polymerase chain reaction mix are combined prior to microdispensing, and are microdispensed together.

Other aspects and advantages of the disclosure will be apparent upon review of the description and drawings.

### BRIEF DESCRIPTION OF DRAWINGS

FIG. 1A is a side cross-sectional schematic view of a microchamber covered by a lid and containing a single cell and liquid.

FIG. 1B is a side cross-sectional schematic view of the microchamber of FIG. 1A following removal of the lid, thereby removing a portion of the liquid from the microchamber.

FIG. 1C is a side cross-sectional schematic view of the microchamber of FIG. 1B arranged proximate to a microdispenser arranged to supply one or more liquids to the microchamber.

FIG. 2 is a side cross-sectional schematic view of a microchamber covered by a lid and containing a single cell, with the microchamber arranged proximate to an optical source and detector.

FIG. 3 is a top plan view illustration of at least a portion of a microarray containing nine microchambers each containing at least one cell.

Features in the figures are not to scale unless specifically indicated to the contrary herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It should be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It should also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

It should be understood that, although the terms “upper,” “lower,” “bottom,” “intermediate,” “middle,” “top,” and the like may be used herein to describe various elements, these elements should not be limited by these terms. These terms are only used to distinguish one element from another. For example, a first element could be termed an “upper” element and, similarly, a second element could be termed an “upper” element depending on the relative orientations of these elements, without departing from the scope of the present disclosure.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having meanings that are consistent with their meanings in the context of this specification and the relevant art and will not be interpreted in an idealized or overly formal sense unless expressly so defined herein.

Disclosed herein is a method for analyzing cells through measurement of live-cell parameters followed by measurement of gene and protein expression. An exemplary method comprises measuring one or more live-cell parameters for a plurality of cells contained in at least one liquid in a plurality of isolated microchambers of a microarray device. The method further comprises removing a lid bounding the plurality of isolated microchambers. The method further comprises microdispensing a quantity of lysate into each microchamber of the plurality of isolated microchambers. The method further comprises microdispensing a quantity of reverse transcription polymerase chain reaction mix into each microchamber of the plurality of isolated microchambers. The method further comprises microdispensing a quantity of oil into each microchamber of the plurality of isolated microchambers. The method further comprises incorporating the microarray device into a thermal cycling apparatus with a window permitting epifluorescence imaging of the plurality of isolated microchambers.

Live-cell parameters can include any number of parameters such as oxygen concentration, oxygen consumption rate, pH, glucose concentration, glucose consumption rate, ATP concentration, and mitochondrial membrane potential. The present disclosure combines measurement of one or more of these parameters with qRT-PCR at the single-cell or multiple-cell level, while maintaining one-to-one correspondence between phenotype and genotype measurements at the microchamber level.

When the live-cell measurements are made using a sensor lid that caps an array of microchambers, sensor lid disassembly will naturally remove a portion of the fluid that was originally present in the sealed microchamber. In certain embodiments, the volume of a microchamber can be 225 pL (e.g., 100 μm diameter and 32 μm deep), of which a single mammalian cell will comprise approximately 4 pL. In certain embodiments, the cell medium is removed to leave a volume of approximately 50 pL. In certain embodiments, microdispensing using conventional piezoelectric droplet dispensing technology is used to deliver chemicals to individual wells with single-well selectivity. In certain embodiments, a dispensed droplet size can be approximately 50 pL or greater. In certain embodiments, the design ratio of lysate to PCR mix is 4:6. In such an embodiment, dispensed lysate volume may be 50 pL, followed by dispensed PCR mix volume of 75 pL. This may be followed by further dispensation of a mineral oil droplet (e.g., 50 pL), which may fill a microchamber having a volume of 225 pL.

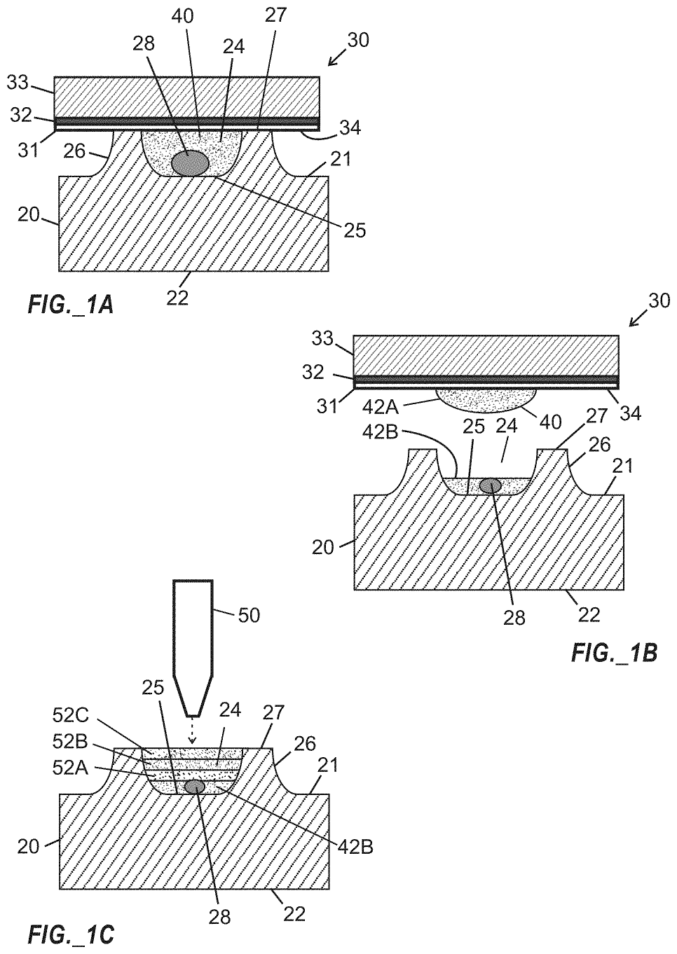

FIG. 1A schematically illustrates a side cross-sectional schematic view of a microchamber 24 covered by a lid 30 (e.g., multi-layer sealing structure) and containing a single cell 28 and at least one primary liquid 40. The microchamber 24 is defined within a recess formed by a lip 26 protruding upward from a substrate 20 of a microfluidic device and a microchamber floor 25. The substrate 20 includes an upper surface 21 and a lower surface 22 that opposes the upper surface 21. In certain embodiments, the lid 30 includes a front compliant layer 31, a flexural layer 32, and a back compliant layer 33, wherein the flexural layer 32 is arranged between the front compliant layer 31 and the back compliant layer 33. In FIG. 1A, the lid 30 is illustrated as assembled with (i.e., above) the substrate 20. When sealed, a lower surface 34 of the lid 30 (including a surface of the front compliant layer 31) may be arranged to contact an upper surface 27 of the lip 26. One or more layers of a microfluidic device, such as a substrate 20 defining a microchamber 20, or a lid 30, may be fabricated of substantially rigid materials such as fused silica, glass, polymers (e.g., molding) and the like. In certain embodiments, the front compliant layer 31, flexural layer 32, and/or back compliant layer 33 comprises an elastomeric material (e.g., rubber, silicone rubber, etc.) and/or metal (e.g., aluminum, etc.).

In certain embodiments, the at least one front compliant layer 31 is substantially impervious to passage of gas (e.g., air) and/or evaporation of contents of a microwell. In certain embodiments, the front compliant layer 31 is optically reflective. In certain embodiments, the front compliant layer 31 comprises a plurality of front compliant layers. In certain embodiments, the front compliant layer comprises a thickness in a range of from 0.06 μm to 100 μm.

In certain embodiments, the back compliant layer 33 comprises an adhesive (e.g., an acrylic adhesive tape or a foam adhesive tape). In certain embodiments, the back compliant layer 33 comprises foam rubber, solid rubber, or silicone rubber. In certain embodiments, a back compliant layer 33 is more compliant than the front compliant layer 31. In certain embodiments, the back compliant layer 33 comprises silicone rubber, e.g., 70 Shore A with an approximate thickness of 0.5 mm. In certain embodiments, the back compliant layer 33 may comprise acrylic Pressure-Sensitive Adhesive (PSA), 50 to 125 μm thick, such as may be embodied or included in transfer tape or double-coated tape. In certain embodiments, the back compliant layer 33 may comprise foam-based tape such as 3M 4016.

In certain embodiments, the flexural layer 32 comprises a polymeric material (e.g., polyethylene terephthalate (PET)). In certain embodiments, the flexural layer 32 comprises a thickness in a range of from 25 μm to 100 μm. In certain embodiments, the flexural layer 32 comprises a plate constant, D, in a range of from 8 kNm to 7000 kNm. In certain embodiments, the flexural layer 32 comprises a modulus of elasticity of at least 1000 MPa.

FIG. 1B illustrates the microchamber 24 of FIG. 1A following removal of the lid 30, whereby a first portion 42A of the at least one primary liquid 40 remains in contact with the lid 30 (e.g., by surface tension) and is thereby removed from the microchamber 24, and a second portion 42B of the at least one primary liquid 40 remains in the microchamber 24 proximate to the cell 28. In FIG. 1B, the lid 30 is illustrated as separated from (i.e., above) the substrate 20. When the lid 30 is removed from the microchamber 24, the lower surface 34 of the lid 30 (including the surface of the front compliant layer 31) is separated from the upper surface 27 of the lip 26.

FIG. 1C illustrates the microwell 24 of FIG. 1B arranged proximate to a microdispenser 50 arranged to supply multiple secondary liquids (e.g., first secondary liquid 52A, second secondary liquid 52B, third secondary liquid 52C) to the microchamber 24, following dispensation of three of the multiple secondary liquids 52A-52C into the microchamber 24. In certain embodiments, the first secondary liquid 52A, second secondary liquid 52B, and third secondary liquid 52C are compositionally different from one another. In certain embodiments, the first secondary liquid 52A comprises lysate, the second secondary liquid 52B comprises a PCR mix volume, and the third secondary liquid 52C comprises mineral oil.

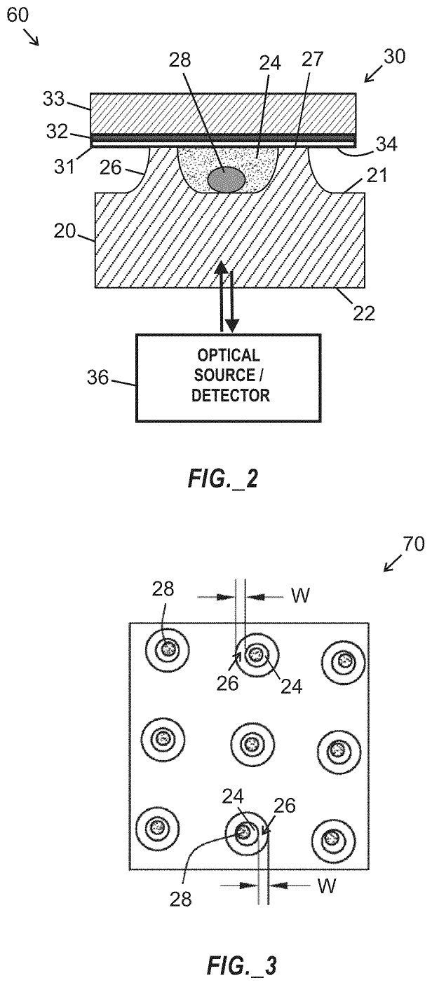

After microdispensation is complete, the microchamber 24 (e.g., as part of a microarray device) may be placed or otherwise incorporated into a thermal cycling apparatus 60 (shown in FIG. 2) enabling performance of qRT-PCR amplification. Preferably, the thermal cycling apparatus 60 includes a window permitting epifluorescence imaging of a plurality of isolated microchambers. FIG. 2 is a side cross-sectional schematic view of a microchamber 24 covered by a lid 30 and containing a single cell 28, with the microwell 24 arranged proximate to an optical source and detector 36. The microchamber 24 is enclosed with the lid 30 contacting a raised lip 26 of a substrate 20 that laterally bounds the microchamber 24. The substrate 20 includes an upper surface 21 and a lower surface 22 that opposes the upper surface 21. The optical source and detector 36 is provided below the lower surface 22 of the substrate 20 proximate to the microchamber 24, with the optical source being arranged to transmit one or more wavelength bands or ranges (e.g., UV emissions, visible light emissions, and/or infrared emissions, including narrow or broad spectral output) into the microchamber 24 to interact with its contents (including the single cell 28), and the optical detector being arranged to receive one or more wavelength bands or ranges following interaction with contents of the microchamber 24. The substrate 20 is preferably transmissive of a broad spectrum of wavelengths, including one or more wavelength ranges identified above. In certain embodiments, fluorescence imaging may be used, in which a range of transmitted wavelengths includes a transmission wavelength peak (e.g., a single wavelength peak), and a range of received wavelengths includes a received wavelength peak (e.g., a single wavelength peak), wherein the range of transmitted wavelengths and the range of received wavelengths may include overlapping or non-overlapping ranges. In certain embodiments, multiple channels may provide independent transmit and receive functions. The lid 30 includes a front compliant layer 31, a flexural layer 32, and a back compliant layer 33, wherein the front compliant layer 31 embodies a lower surface 34 of the lid 30. In an embodiment wherein the lid 30 includes an optically reflective layer, such optically reflective layer may desirably reflect light to the detector portion of the optical source and detector 36.

Although FIGS. 1A-2 each illustrate a single microchamber, a microarray device including an array of microchambers 24 is contemplated. FIG. 3 is a top plan view illustration of at least a portion of a microarray 70 containing nine microchambers 24 each bounded by a lip 26 (having a width w) and each containing at least one cell 28. A microarray device 70 may include any suitable or desirable number of microchambers 24. In certain embodiments, the number of microchambers 24 may be more than 50, more than 100, more than 500, or more than 1000.

In certain embodiments, the total fluid volume dispensed in a microchambers 24 can exceed the well volume, resulting in excess fluid which, if the volume is of reasonable size, will not run away. A positive control is an RNA spike (e.g., 1 nanogram of control RNA per well in 50 pL of aqueous volume either with or without a cell) because the spike is much more concentrated than a cell. This is followed by one step qRT-PCR mix and then the oil droplet. One negative control is a no-primer control in which the primer is omitted. Another negative control could be wells with no cells.

By the nature of random seeding, some wells 24 will be empty if the seeding density is selected appropriately. Empty wells 24 are used for positive and negative controls. The exact locations of empty wells 24 can be detected prior to PCR and then used to control the droplet program, or the controls can be dispensed in a fixed manner in which case some controls would be in wells with cells and some controls would be in wells without cells. A well 24 with no cell and no primer is a well that is not used. Statistically, there will almost always be the required negative controls in each assay. In certain embodiments utilizing micro qRT-PCR, the following 4 chemicals may be dispensed: lysate; qRT-PCR mix with primers; qRT-PCR mix without primers; and oil. Piezoelectric droplet dispense can be integrated into this process by transporting the well substrate (with live cells) to a dedicated programmable droplet dispense machine (e.g., a Rainmaker MicroDispensing Pattern Generator, available from Engineering Arts, Tempe, Ariz.). Alternatively, dispensation can be accomplished by integrating standard ink-jet printer-type piezo droplet dispensing into the fixtures and equipment used to make metabolic measurements on the cells earlier in the process. (An example of such equipment is described in Kelbauskas, et al., “Method for physiologic phenotype characterization at the single-cell level in non-interacting and interacting cells.” J. of Biomed. Opt., 17, 037008 (2012)). In the latter case, the lid used for sealing the chamber is removed and replaced with the piezoelectric dispensing head (e.g., arranged for non-contact, but close proximity dispensing relative to the wells). This is convenient because the same equipment used to perform fluorescence imaging for metabolic analysis can be used for qRT-PCR measurement.

In another embodiment, the oil is dispensed by spraying rather than by piezoelectric dispensation. The accuracy of the ratio of volumes of lysate to qRT-PCR mix by piezoelectric tip dispensing can easily be calibrated by dispensing two separate controls for which output is very sensitive to ratio.

In an additional embodiment, in-cell Western Blot for protein detection is accomplished within individual wells using the same microdispensing technology. At the chip level, formaldehyde is dispensed to fix the cells in place in the microchambers followed by wash, then permeabilization of cell membranes with Triton X-100, then wash, then blocking solution. Subsequently, specific primary antibodies, and then secondary antibodies, can be microdispensed at the individual well level in a manner similar to that described above. This can be achieved using different and selectable antibodies from well to well, or using multiplexing within an individual well. This has the advantages of maintaining measurement independence between microchambers, achieving high throughput, and minimizing the volume of expensive antibodies.

Upon reading the foregoing description in light of the accompanying drawing figures, those skilled in the art will understand the concepts of the disclosure and will recognize applications of these concepts not particularly addressed herein. Those skilled in the art will recognize improvements and modifications to the preferred embodiments of the present disclosure. All such improvements and modifications are considered within the scope of the concepts disclosed herein and the claims that follow.

What is claimed is:

1. A method for analyzing cells, the method comprising:
measuring one or more live-cell parameters for a plurality of cells and at least one liquid distributed among a plurality of isolated microchambers of a microarray device;
removing a multi-layer lid bounding the plurality of isolated microchambers, the multi-layer lid comprising a front compliant layer, a flexural layer, and a back compliant layer, wherein the flexural layer is arranged between the front compliant layer and the back compliant layer, the front compliant layer is closer than the back compliant layer to the plurality of microchambers, and an entirety of a surface of the front compliant layer is optically reflective;
microdispensing a quantity of lysate into each microchamber of the plurality of isolated microchambers;
microdispensing a quantity of reverse transcription polymerase chain reaction mix into each microchamber of the plurality of isolated microchambers;
microdispensing a quantity of oil into each microchamber of the plurality of isolated microchambers;
after said microdispensing steps, covering the plurality of microchambers with the multi-layer lid, with the front compliant layer in contact with the microarray device; and
incorporating the microarray device into a fixture incorporating a thermal cycling apparatus as well as an optical source, an optical detector, and a window permitting epifluorescence imaging of the plurality of isolated microchambers, wherein the optical detector is configured to measure fluorescence response of each microchamber of the plurality of isolated microchambers.

2. The method of claim 1, wherein each microchamber of the plurality of isolated microchambers comprises a volume in a range of from about 100 picoliters to about 500 picoliters.

3. The method of claim 1, wherein said removing of the multi-layer lid bounding the plurality of isolated microchambers causes removal of a portion of the at least one liquid from the plurality of isolated microchambers.

4. The method of claim 1, further comprising removing a portion of the at least one liquid from the plurality of isolated microchambers by at least one of evaporation, blotting, or application of a gas flow.

5. The method of claim 1, further comprising microdispensing a quantity of control RNA into a subset of microchambers of the plurality of isolated microchambers.

6. The method of claim 1, wherein at least one of said microdispensing of a quantity of lysate, microdispensing of a quantity of reverse transcription polymerase chain reaction mix, or microdispensing of a quantity of oil comprises piezoelectric microdispensing.

7. The method of claim 1, wherein at least one of said quantity of lysate, said quantity of reverse transcription polymerase chain reaction mix, or said quantity of oil comprises a volume in a range of from about 25 picoliters to about 200 picoliters.

8. The method of claim 1, wherein at least one of said quantity of lysate, said quantity of reverse transcription polymerase chain reaction mix, or said quantity of oil comprises a volume in a range of from about 50 picoliters to about 150 picoliters.

9. The method of claim 1, wherein each microchamber of the plurality of isolated microchambers contains a single cell of the plurality of cells.

10. The method of claim 1, wherein said measuring of one or more live-cell parameters is performed at a single-cell level.

11. The method of claim 1, further comprising performing a reverse transcription polymerase chain reaction in each microchamber of the plurality of isolated microchambers.

12. The method of claim 11, further comprising performing protein expression measurement at a single-cell level in each microchamber of the plurality of isolated microchambers.

13. The method of claim 1, wherein at least one of said microdispensing steps is performed with at least one piezoelectric dispensing head.

14. The method of claim 1, wherein said one or more live-cell parameters comprise at least one of oxygen concentration, oxygen consumption rate, pH, glucose concentration, glucose consumption rate, adenosine triphosphate concentration, or mitochondrial membrane potential.

15. The method of claim 1, wherein said quantity of lysate and said quantity of reverse transcription polymerase chain reaction mix are combined prior to microdispensing, and are microdispensed together.

16. The method of claim 1, wherein the back compliant layer is more compliant than the front compliant layer.

17. The method of claim 1, wherein the back compliant layer comprises (i) an elastomeric material or foam material, and (ii) an adhesive material.

18. The method of claim 1, wherein the flexural layer comprises a polymeric material and has a modulus of elasticity of at least 1000 MPa.

19. The method of claim 1, wherein:
one or more of the front compliant layer and the back compliant layer comprises an elastomeric material and/or a metal;
the back compliant layer is more compliant than the first compliant layer; and
the flexural material comprises a polymeric material.

20. The method of claim 19, wherein:
the front compliant layer comprises a thickness in a range of from 0.06 μm to 100 μm;
the back compliant layer comprises a thickness greater than the front compliant layer; and
the flexural layer comprises a thickness in a range of from 25 μm to 100 μm.
